(19) United States
(12) Patent Application Publication
(10) Pub. No.: US 2008/0014272 A1
(43) Pub. Date: Jan. 17, 2008

(54) Compositions and Methods for Treatment of Chronic Pain Conditions

(76) Inventors: Phil Skolnick, Edgewater, NJ (US); Warren Stern, Plymouth, MA (US)

Correspondence Address: Jeffrey J. King, Esq.;BLACK LOWE & GRAHAM PLLC, Suite 4800, 701 Fifth Avenue, Seattle, WA, 98104, US

(21) Appl. No.: 11/775,721

(22) Filed: Jul. 10, 2007

Related U.S. Application Data
(60) Provisional application No. 60/830,412, filed on Jul. 11, 2006.

(51) Int. Cl. A61K 31/403; A61K 31/19; A61K 31/44; A61K 9/00; A61P 19/00; A61P 19/02

(52) U.S. Cl. 424/484; 514/282; 514/415; 514/557

(57) ABSTRACT

The present invention relates to methods, pharmaceutical compositions and kits for treating osteoarthritis associated pain, inflammation and improving function in a patient comprising a first therapeutic agent which comprises bicifadine or a pharmaceutically acceptable salt, enantiomer, solvate, hydrate, polymorph or prodrug thereof and a second therapeutic agent which comprises a non-steroidal anti-inflammatory drug or derivative thereof.

### RELATED APPLICATIONS

This application claims all priority benefits of U.S. Provisional patent application No. 60/830,412, filed Jul. 11, 2006 which is incorporated herein by reference.

### TECHNICAL FIELD

The present invention relates to compositions and methods for treatment of chronic pain conditions, including osteoarthritis, rheumatoid arthritis, chronic low back pain, and other chronic pain conditions. More specifically, the present invention relates to compositions and methods to alleviate chronic pain and/or to reduce functional disabilities associated with chronic pain.

### BACKGROUND OF THE INVENTION

Chronic pain conditions are diverse and widespread in humans and other subjects, often causing serious impacts on quality of life and resulting in serious disabilities including functional and activity impairment. The high prevalence and morbidity of chronic pain conditions result in high costs for human and veterinary health care to treat chronic pain. Exemplary chronic pain conditions for which new drugs and therapies are urgently needed include osteoarthritis pain; rheumatoid arthritis pain; cancer pain; and various other chronic pain conditions of non-neuropathic origin, such as chronic low back pain, chronic lumbar and cervical pain, chronic fibromyalgia pain, chronic pain from arteriovenuous malformation, arachnoiditis, chronic pain from root avulsion, chronic postthoracotomy pain, and chronic postmastectomy pain of non-neuropathic origin.

Osteoarthritis (OA), sometimes called degenerative joint disease, hypertrophic arthritis, degenerative arthritis or osteoarthrosis is the most common form of arthritis currently afflicting more than 21 million Americans. It is the most common rheumatic disease affecting humans and it is the second most common disease responsible for disability in the United States. (Peyron J G, Altman R D: The Epidemiology of Osteoarthritis. In Osteoarthritis: Diagnosis and Medical/Surgical Management, edn 2. Edited by Moskowitz R W, Howell D S, Goldberg V M. Philadelphia: W B Saunders; 1992:15-37). Despite the frequency of the disease, the cause is still unknown and there is no cure or effective treatment.

OA is a chronic and progressive disease which causes damage to afflicted joints and surrounding tissues. It often develops slowly as the articular cartilage that lies on the ends of bones and forms the articulating surface of the joints gradually degenerates. As cartilage degeneration progresses, secondary changes occur in other tissues in and around the joints including the bone, muscle, ligaments, menisci and synovium. The effect of the failure of cartilage tissue and secondary damage to other tissues is that an individual experiences pain, swelling, weakness and loss of functional ability in the afflicted joint(s). Individuals with OA have pain that typically worsens with weight bearing and activity and improves with rest. Many individuals with OA develop functional/activity impairments, such as impaired coordination, and postural and gait defects due to pain and stiffness. These symptoms frequently progress to a point where quality of life, functional capabilities, productivity and other functional/activity measures are profoundly affected.

Generally, OA is treated pharmacologically. However, currently approved pharmacological agents such as acetaminophen or non-steroidal anti-inflammatory drugs (NSAIDs), such as aspirin, ibuprofen, and cyclo-oxygenase-2 (COX-2) inhibitors, only reduce pain by approximately 30 percent. (Schnitzer T J. Update of ACR guidelines for osteoarthritis: role of coxibs. J. Pain Symptom Manage. 2002; 23:S24-30; Easton B T. Evaluation and treatment of the subject with osteoarthritis. J. Family. Practice. 2001; 50: 791-797; Chard J, Dieppe P. Update: treatment of osteoarthritis. Arthritis and Rheumatism. 2002; 47: 686-690.) Steroids may be used to treat swelling caused by OA, however, steroids are contraindicated for long term use due to side effects such as increased bone loss and increased susceptibility to infection. Surgery such as joint replacement, arthroscopic lavage and debridement, repositioning of bones or fusing of bones may also be recommended. However, surgery, particularly in older patients, has increased risks of complications and long recovery periods.

There is therefore an urgent need in the art for new and alternative treatments for management of chronic pain, including osteoarthritis, and for alleviating disabilities, including function/activity impairment, in patients with chronic pain.

### SUMMARY OF EXEMPLARY EMBODIMENTS OF THE INVENTION

The present invention provides methods and compositions to treat and/or prevent chronic pain conditions in mammalian subjects. The compositions and methods of the invention effectively treat a wide variety of chronic pain conditions and symptoms, including, for example, osteoarthritis (OA) pain; rheumatoid arthritis pain; cancer pain; and various other chronic pain conditions of non-neuropathic origin, including chronic low back pain, chronic lumbar and cervical pain, chronic fibromyalgia pain, chronic pain from arteriovenuous malformation, arachnoiditis, chronic pain from root avulsion, chronic postthoracotomy pain, and chronic postmastectomy pain of non-neuropathic origin. The methods and compositions of the invention employ combinatorial formulations and/or coordinate treatment methods wherein an effective amount of bicifadine is administered to a patient suffering from chronic pain in combination with one or more non-steroidal anti-inflammatory drugs (NSAIDs), wherein the combination of drug treatments is sufficient to alleviate one or more targeted chronic pain condition(s) or symptom(s).

Within certain embodiments of the invention, methods for treating chronic pain associated with OA are provided which employ an effective combination of bicifadine and one or more NSAID(s) sufficient to alleviate one or more condition(s) or symptom(s) of OA.

In other embodiments, the methods and compositions of the invention employing bicifadine in combination with one or more NSAID(s) are effective for inhibiting progression of a chronic pain condition in a mammalian subject.

In a further embodiment, the invention is directed to methods and compositions for treating inflammation associated with a chronic pain condition, such as inflammation associated with OA. These methods similarly employ a combination of bicifadine and one or more NSAID(s) sufficient to alleviate one or more symptoms of inflammation associated with a chronic pain condition.

In another embodiment, the invention is directed to methods and compositions for treating or preventing a disability associated with chronic pain, for example by reducing or eliminating functional/activity impairment in a patient suffering from chronic pain. These methods and compositions likewise employ a combination of bicifadine and one or more NSAID(s) effective to treat or prevent a targeted disability in a patient suffering from chronic pain.

As used herein, “bicifadine” refers to the drug bicifadine as described herein, including all pharmaceutically acceptable salts, enantiomers, solvates, hydrates, polymorphs and prodrugs thereof.

Bicifadine, whose chemical name is (±)-1-(4-methyl phenyl)-3-azabicyclo[3.1.0]hexane and whose synonym is racemic 1-(ρ-tolyl)-3-azabicyclo[3.1.0]hexane, is a member of a series of 1-(substituted phenyl)-3-azabicyclo[3.1.0]hexanes. This series of compounds has been previously described (See, for example, U.S. Pat. Nos. 4,088,652, 4,118,393, 4,118,417, 4,131,611, 4,196,120 and 4,231,935). Bicifadine has been used to treat nociceptive pain, however, as explained in further detail below, the inventors have determined that bicifadine by itself is ineffective in the treatment of osteoarthritic pain.

Useful forms of bicifadine within the formulations and methods of the invention include the compounds described herein, as well as their active pharmaceutically acceptable salts, enantiomers, polymorphs, solvates, hydrates and/or prodrugs, or combinations thereof.

Non-steroidal anti-inflammatory drugs (NSAIDs) are compounds that inhibit the activity of one or both Cox-1 and Cox-2 enzymes. Both cox enzymes produce prostaglandins that promote inflammation, pain, and fever. NSAIDs block the Cox enzymes and reduce prostaglandins throughout the body thereby reducing inflammation, pain, and fever.

Useful NSAIDs within the formulations and methods of the invention include, but are not limited to, salicylates including, but not limited to, aspirin, aloxiprin, salsalate, choline magnesium trisalicylate, diflunisal, salicylaide, salicylic acid, choline salicylates, sodium salicylate, triethanolamine salicylate, magnesium salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib, and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as their active pharmaceutically acceptable salts, enantiomers, polymorphs, solvates, hydrates and/or prodrugs, or combinations thereof.

Useful tertiary or adjunctive agents within the formulations and methods of the invention include all known drugs and agents which are effective in relieving osteoarthritis and pain and inflammation caused by osteoaithritis. Useful tertiary or adjunctive agents in this context include, but are not limited to, topical pain relievers including, but not limited to those containing methyl salicylate, menthol, camphor, eucalyptus and capsaicin; tramadol; acetaminophen; glucosamine; allopurinol; colchicine; demecolcine; oxypurinol; chondroitin; corticosteroid injections, including but not limited to glucocorticoids; and hyaluronic acid derivatives, including, but not limited to sodium hyaluronate and hylan G-F20. Adjunctive therapies may also be used including, but not limited to, physical treatments such as changes in diet, exercise, weight loss, heat treatment, cold treatment, acupuncture and surgery including, but not limited to, joint replacement, osteotomy, arthroscopic lavage and debridement, repositioning of bones, bone fusion, discectomy, and spinal fusion.

In exemplary embodiments, the compositions and methods of the invention employ bicifadine in combination with one or more NSAID(s) to treat chronic pain, for example OA. Surprisingly, bicifadine, which by itself is ineffective in treating osteoarthritic pain, demonstrates a combinatorial effectiveness for treating osteoarthritic pain and improving function when administered in conjunction with one or more NSAID(s). The combination of bicifadine (including pharmaceutically acceptable salts, enantiomers, solvates, hydrates, polymorphs and prodrugs of bicifadine) administered coordinately with a NSAID yields an enhanced therapeutic response beyond the therapeutic response elicited by either bicifadine or the NSAID alone. As a result, in certain embodiments of the invention the dosage of a NSAID required to adequately treat chronic pain (i.e., to significantly reduce one or more chronic pain condition(s) or symptom(s)) as contemplated herein, may be reduced compared to an effective dose of the NSAID alone for treating the subject pain condition. In related embodiments, lower doses of bicifadine and NSAIDs employed within the invention reduce the occurrence and severity of side-effects elicited by higher doses of bicifadine and/or the NSAID alone to treat the targeted chronic pain condition. Additionally, in certain embodiments of the invention, doses of bicifadine and a NSAID required to adequately treat chronic pain and/or improve activity/function in subjects with chronic pain, such as OA, may each be below a usual threshold of the corresponding drug for maximal analgesic efficacy for treatment of the targeted chronic pain condition.

Animal subjects amenable for treatment according to the formulations and methods of the invention include, but are not limited to, human and other mammalian subjects suffering from chronic pain. In exemplary embodiments, subjects amenable for treatment using the methods and compositions of the invention will present with a chronic pain-related disability, such as one or more functional/activity impairments or limitations associated with chronic pain.

These and other subjects are effectively treated prophylactically and/or therapeutically, by administering to the subject an effective amount of bicifadine in combination with one or more NSAID(s). As noted above, the methods and formulations of the present invention may employ bicifadine and/or the NSAID(s) in a variety of forms including pharmaceutically acceptable salts, enantiomers, polymorphs, solvates, hydrates and/or prodrugs or combinations thereof. In one exemplary embodiment herein, ibuprofen is employed as an illustrative NSAID to effectively treat chronic pain in combination with bicifadine.

Within certain embodiments of the invention, distinct methods for preventing or treating a condition or symptom of chronic pain in mammalian subjects are provided which involve administering to the subject an effective amount of bicifadine in a daily dosing regimen consisting of only one or two doses of bicifadine per day in a coordinate regimen with one or more NSAID(s) (wherein the NSAID(s) may be administered simultaneously or in various independent dosing regimens). This limited dosing regimen for bicifadine is surprisingly effective in combination with the NSAID to alleviate or prevent chronic pain conditions in human and other subjects, without attendant, unacceptable adverse side effects, over an extended period, e.g., up to a 24 hour period. Within alternate embodiments of the invention, a once daily or twice daily dosing protocol for bicifadine is provided which employs either an immediate release, controlled release, or sustained release bicifadine formulation, which is effective in a coordinate treatment protocol for treating the chronic pain over an extended period (wherein the coordinate dosing schedules for the coordinately administered NSAID(s) may be the same, or independent of the bicifadine dosing schedule). Within certain exemplary embodiments, a sustained release composition or dosage form of bicifadine is employed in a method for treating chronic pain involving a limited bicifadine dosing schedule of once or twice daily administration, wherein an incidence and/or severity of one or more adverse side effects is reduced in treatment subjects compared to an incidence and/or severity of the same side effect(s) observed in subjects after administration of an equivalent amount of bicifadine in an immediate release bicifadine formulation. Additional discussion of bicifadine dosing, formulation, and other subjects related to the instant invention is provided, for example, in U.S. patent application Ser. No. 11/438,909, filed May 22, 2006; U.S. patent application Ser. No. 11/260,887, filed Oct. 26, 2005, U.S. patent application Ser. No. 10/621,435, filed Jul. 17, 2003; and U.S. Provisional Application No. 60/399,852, filed Jul. 31, 2002, each of which disclosures is incorporated herein by reference for all purposes.

Within exemplary embodiments of the invention, bicifadine and one or more NSAID(s) are combinatorially formulated or coordinately administrated to effectively treat one or more symptoms or conditions of OA in human subjects.

Within further aspects of the invention, combinatorial formulations and methods are provided which employ an effective amount of bicifadine and one or more NSAID(s), and optionally including one or more tertiary or adjunctive therapeutic agents combinatorially formulated or coordinately administered with bicifadine and the NSAID(s) to treat or prevent one or more symptoms of chronic pain in a mammalian subject. Exemplary combinatorial formulations and coordinate treatment methods in this context employ bicifadine and one or more NSAID(s) in combination with none, one, or multiple, tertiary and/or adjunctive active agent(s) that may be combinatorially formulated or coordinately administered with the bicifadine and NSAID(s), to effectively treat a chronic pain condition in a mammalian subject. The adjunctive therapeutic agents or methods used in conjunction with bicifadine and a NSAID in these embodiments may possess direct or indirect effects to alleviate chronic pain, alone or in combination with the bicifadine and NSAID, or may exhibit other useful adjunctive therapeutic activity in combination with the bicifadine and NSAID.

### BRIEF DESCRIPTION OF DRAWINGS

FIG. 1 is a chart depicting a comparison of the effect of morphine versus bicifadine in the treatment of osteoarthritis induced in rats by monoiodoacetamide.

FIG. 2 is a chart depicting patients' assessments of the change in chronic osteoarthritic pain from baseline just prior to administration of the first dose to 3 hours post dose on the final day of each Treatment Period.

FIG. 3 is a chart of the change in patients' WOMAC Osteoarthritis Index scores prior to first morning dose on first treatment day, and at 3 h post-dose on the last day of each Treatment Period,

FIG. 4 is a chart of Global Assessment of Study Medication assessment at 3 h post-dose on the last day of each Treatment Period.

### DETAILED DESCRIPTION OF EXEMPLARY EMBODIMENTS OF THE INVENTION

The present invention provides methods and compositions, including novel compositions, dosage forms and methods of administration, useful for treating and/or preventing chronic pain conditions and associated symptoms (e.g., inflammation or functional disability). The subject methods and compositions employ bicifadine coordinately administered with one or more non-steroidal anti-inflammatory drugs (NSAIDs). In exemplary embodiments, the methods and compositions of the invention alleviate one or more conditions or symptoms of osteoarthritis (OA), for example by reducing pain, stiffness, swelling, inflammation, or tenderness in a patient presenting with OA and/or by reducing disability or improving physical function or activity in an OA patient (e.g., by increasing mobility). The invention additionally provides methods and compositions for inhibiting progression of one or more chronic pain condition(s) or symptom(s).

Formulations and methods within the invention employ bicifadine and one or more NSAID(s) for treating or preventing chronic pain, as exemplified by OA. Within these formulations and methods, the bicifadine and NSAID may be provided in any of a variety of forms, including all active pharmaceutically acceptable salts, solvates, hydrates, polymorphs, enantiomers, and prodrugs, or combinations thereof. Within the formulations and methods of the invention, the bicifadine and NSAID are administered combinatorially or coordinately to effectively treat mammalian subjects suffering from chronic pain, such as OA pain, including one or more attendant symptom(s) associated with chronic pain (such as pain, tenderness, swelling, stiffness, inflammation and functional impairment/disabilities commonly experienced by chronic pain sufferers).

In more detailed embodiments, bicifadine hydrochloride is employed within the therapeutic compositions and methods of the present invention. Bicifadine hydrochloride exists in at least two polymorphic crystalline forms designated polymorph forms A and B (e.g., as described in U.S. patent application Ser. No. 10/702,397, herein incorporated by reference). Other polymorphic forms of bicifadine hydrochloride may also exist and are likewise candidates for use for treating osteoarthritis within the compositions, methods and kits of the present invention.

Polymorphs include compounds with identical chemical structure but different crystalline solid forms. Additionally, many pharmacologically active organic compounds regularly crystallize incorporating second, foreign molecules, especially solvent molecules, into the crystal structure of the principal pharmacologically active compound forming pseudopolymorphs. When the second molecule is a solvent molecule, the pseudopolymorphs can also be referred to as solvates. All of these additional forms of bicifadine are likewise useful within the methods and formulations of the present invention.

Polymorph form A of bicifadine hydrochloride can be formed, for example, by methods disclosed in U.S. Pat. No. 4,231,935 and U.S. Pat. No. 4,196,120 (each of which is incorporated herein by reference). Polymorph form B can be formed, for example, by methods disclosed in U.S. patent application Ser. No. 10/702,397, related international application PCT/US2003/035099 (Intl. Pub. No. WO04/043920), and priority U.S. Provisional Patent Application No. 60/424,982 (each incorporated by reference herein). For example, polymorph form B can be formed from polymorph form A through the application of kinetic energy and through crystallization techniques. In one embodiment, kinetic energy in the form of agitating, stirring, grinding or milling can be applied to a pure composition of polymorph form A, or a mixture of forms A and B, particularly at selected temperatures, for example from about −200° C. to about 50° C., in another embodiment from about −200° C. to about 35° C., in a further embodiment from about −200° C. to about 0° C. In another embodiment, polymorph form B can be crystallized from a solution of polymorph form A that is heated and allowed to cool under defined conditions of temperature and time to form polymorph form B. Under selected conditions, preparations of pure polymorph form A of bicifadine, or mixtures of polymorph forms A and B of bicifadine, can be processed to yield desired compositions containing enriched quantities of polymorph B, for example ranging from approximately at least 10%, to about 10-20%, 20-35%, 35-50%, 50-70%, 70-85%, 85-95%, and up to 95-99% or greater (by weight) bicifadine polymorph form B in the composition. The polymorphs of bicifadine HCl may be characterized by their infrared spectra and/or their x-ray powder diffraction pattern as described in U.S. patent application Ser. No. 10/702,397, filed Nov. 5, 2003, and corresponding United States Patent Publication 20040102638 A1, published May 27, 2004 (incorporated by reference herein).

The bicifadine utilized in the compositions and methods of the present invention for treating chronic pain may comprise any crystalline polymorphic or amorphous form of the compound, or mixture(s) thereof. In exemplary embodiments, the bicifadine utilized in effective therapeutic dosage forms for treating mammalian subjects suffering from chronic pain, for example OA, will comprise essentially pure bicifadine hydrochloride polymorph form A (i.e., having a concentration of 90-95% form A by weight of total bicifadine present), essentially pure polymorph form B, or any mixture of polymorph forms A and B. In certain embodiments, the bicifadine utilized in the compositions, methods and kits of the present invention may contain from about 10% to 98% polymorph form B. In other embodiments, the bicifadine utilized in the compositions of the present invention may be present as greater than about 50% polymorph form B, greater than about 75% polymorph form B, or greater than about 90% polymorph form B.

In additional embodiments, one or more isolated (+) or (−) enantiomers of bicifadine are employed within the compositions and methods of the invention for treating chronic pain. The (+) and (−) enantiomers of bicifadine, and methods for resolving these enantiomers to yield essentially pure compositions of the respective enantiomers, are reported by Epstein et al. (J. Med. Chem. 24(5:481, 1981; NIDA Res. Monogr. pp. 93-98, 1982) and incorporated by reference herein. See also, U.S. Pat. No. 4,131,611; U.S. Pat. No. 4,118,417; U.S. Pat. No. 4,196,120; U.S. Pat. No. 4,231,935; and U.S. Pat. No. 4,435,419, each incorporated herein by reference. In exemplary embodiments, the bicifadine utilized in effective therapeutic dosage forms for treating mammalian subjects presenting with chronic pain will comprise essentially pure (+) bicifadine (i.e., having a concentration of 90-95% of the (+) enantiomer by weight of total bicifadine present), essentially pure (−) bicifadine, or any racemic mixture of the (+) and (−) enantiomeric forms of bicifadine. In certain embodiments, the bicifadine utilized in the composition may contain from about 10% to 98% (+) or (−) bicifadine. In other embodiments there may be present in bicifadine utilized in the formulation greater than about 50% (+) or (−) bicifadine, greater than about 75% (+) or (−) bicifadine, or greater than about 90% (+) or (−) bicifadine.

As noted above, in certain embodiments the methods and compositions of the invention may employ pharmaceutically acceptable salts, e.g., acid addition or base salts of the above-described bicifadine and NSAID compounds and/or related or derivative compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, taitrate, and gluconate salts may also be formed. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like. Suitable base salts are formed from bases that form non-toxic salts, for example aluminum, calcium, lithium, magnesium, potassium, sodium, zinc and diethanolamine salts, sulfate, citrate, acetate, oxalate, chloride, bromide, iodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

In other detailed embodiments, the methods and compositions of the invention employ prodrugs of bicifadine and/or a NSAID. Prodrugs are considered to be any covalently bonded carrier which releases the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions comprising a compound or derivative compound of bicifadine and a NSAID using in vivo metabolic products of the said compounds (either generated in vivo after administration of the subject precursor compound, or directly administered in the form of the metabolic product itself). Such products may result, for example, from the oxidation, reduction, hydrolysis, amidation, esterification and the like of the administered compound, primarily due to enzymatic processes. Accordingly, the invention includes methods and compositions of the invention employing compounds produced by a process comprising contacting a compound or derivative compound of bicifadine and a NSAID with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

Within the methods and compositions of the invention, bicifadine and NSAIDs or their derivatives are effectively formulated and administered to treat chronic pain. In exemplary embodiments, ibuprofen is used as an illustrative NSAID coordinately administered with bicifadine. Other NSAIDs which may also be effectively employed in combinatorial formulations and coordinate treatment methods of the invention to treat chronic pain include, but are not limited to, salicylates including, but not limited to, aspirin, aloxiprin, salsalate, choline magnesium trisalicylate, diflunisal, salicylamide, salicylic acid, choline salicylates, magnesium salicylate, sodium salicylate, triethanolamine salicylate, flufenisal, benorylate, and fisalamine; arylalkanoic acids including, but not limited to, diclofenac, aclofenac, indomethacin, desoxysulindac and sulindac; N-arylanthranilic acids (fenamic acids) including, but not limited to, mefenamic acid, flufenamic acid, and meclofenamate sodium; oxicams including, but not limited to piroxicam, tenoxicam, meloxicam, lomoxicam and tesicam; coxibs including, but not limited to, celecoxib, rofecoxib, valdecoxib, parecoxib and etoricoxib; sulphonanilides including, but not limited to, nimesulide; napthylalkanones including, but not limited to, nabumetone; acetic acids including, but not limited to, diclofenac, ibufenac, fenbufen, indomethacin, indoxole, sulindac, etoldac, and tolmetin; propionic acids including, but not limited to, oxaprozin, ibuprofen, flurbiprofen, oxaprozin, ketoprofen, naproxen, naproxol, carprofen, fenoprofen, fluprofen, and ketorolac; sulfonamides including, but not limited to, trifumidate; pyrazoles including, but not limited to, phenylbutazone, aminopyrine, antipyrine, oxyphenbutazone, and tetrydamine; aminonicotinic acids including, but not limited to, flunixin; pyrazolones including, but not limited to phenylbutazone, feprazone, and apazone. Additional NSAIDs which may be used in the present invention further include, but are not limited to, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide, as well as combinations of the foregoing, and their active pharmaceutically acceptable salts, enantiomers, polymorphs, solvates, hydrates and/or prodrugs.

A broad range of mammalian subjects, including human subjects, are can be effectively treated for chronic paint conditions using the formulations and methods of the invention. These subjects include, but are not limited to, human and other mammalian subjects suffering from chronic pain, for example chronic low back pain (CLBP) or pain associated with OA.

Within the methods and compositions of the invention, bicifadine and one or more NSAID(s) are effectively formulated or coordinately administered together to treat osteoarthitis and related conditions in mammals. In exemplary embodiments, the combination of bicifadine and a NSAID is shown to be effective in pharmaceutical formulations and methods. It is further apparent from the present disclosure that additional pharmaceutically acceptable bicifadine and a non-steroidal anti-inflammatory compounds, complexes, salts, polymorphs, enantiomers, solvates, hydrates and/or prodrugs, or combinations thereof will be comparably effective in treating osteoarthritis within the methods and compositions of the invention.

Compositions of the invention for treating chronic pain may comprise an effective amount of bicifadine and one or more NSAID(s) which is effective for the treatment or prevention of the targeted pain condition and/or one or more related conditions or symptoms. Typically, effective amounts of bicifadine and a NSAID will comprise amounts of each active compound which are therapeutically effective, alone or in combination, in a single or multiple unit dosage form, over a specified period of therapeutic intervention, to measurably alleviate one or more symptom(s) of a targeted chronic pain condition, such as OA, in the subject. The active compounds may be optionally formulated with a pharmaceutically acceptable carrier and/or various excipients, vehicles, stabilizers, buffers, preservatives, etc.

The methods and compositions of the invention for treating chronic pain may further compromise a tertiary or adjunctive agent or therapy. Useful tertiary or adjunctive agents and therapies within the formulations and methods of the invention include all drug agents and therapeutic methods which are effective in relieving a targeted chronic pain condition, such as OA, as well as symptoms associated with the chronic pain, such as pain, inflammation, functional disability, etc. Useful tertiary or adjunctive agents in this context include, but are not limited to, additional NSAIDs, all classes of analgesics, including opiates, topical pain relievers including, but not limited to those containing methyl salicylate, menthol, camphor, eucalyptus and capsaicin; tramadol; acetaminophen; glucosamine; allopurinol; colchicine; demecolcine; oxypurinol; chondroitin; corticosteroid, and hyaluronic acid derivatives. Adjunctive therapies within the methods and compositions of the present invention include, but are not limited to, physical treatments such as changes in diet, exercise, weight loss, heat treatment, cold treatment, acupuncture and surgery including, but not limited to, joint replacement, osteotomy, arthroscopic lavage and debridement, repositioning of bones, bone fusion, discectomy, and spinal fusion.

The amount, timing and mode of delivery of compositions of the invention comprising an anti-chronic pain effective amount of bicifadine and one or more NSAID(s) will be routinely adjusted on an individual basis, depending on such factors as weight, age, gender, and condition of the individual, the acuteness of the chronic pain and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant anti-osteoarthritic formulations will ordinarily be selected to approximate a minimal dosing regimen that is necessary and sufficient to substantially prevent or alleviate osteoarthritis and related conditions in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

Within one embodiment of the invention, it has been surprisingly found that conditions and symptoms of chronic pain in mammalian subjects can be effectively treated by administering to the subject a therapeutically effective amount of an active therapeutic agent selected from bicifadine (including pharmaceutically acceptable salts, enantiomers, polymorphs, solvates, hydrates, and prodrugs of bicifadine), in a daily dosing regimen consisting of only one or two doses of the active agent per day. Based on findings from extensive studies employing bicifadine HCl for treating acute pain (for example acute dental pain and bunionectomy pain studies), exemplary unit doses (e.g., in the range of about 200 mg, 400 mg, or 600 mg) of bicifadine, yielding acceptable side effect levels, were found to have a period of analgesic efficacy for effectively treating acute pain of approximately 6 hours or even less. Duration of analgesic efficacy of bicifadine in acute dental pain studies was estimated using a “last observation carried forward” (LOCF) statistical method, and was projected to last up to 6 hours, or even up to 12 hours (see, e.g., Czobor P., et al., Stark J., Beer G., Petti S., Lippa A., Brown J., Beer B.: A Double-Blind, Placebo Controlled Randomized Study of DOV220,075 (bicifadine) SR and Codeine 60 mg in the Treatment of Post-Operative Dental Pain. Presented at the 2nd Annual Scientific Meeting Mar. 20-23, 2003 Chicago, Ill. American Pain Society Abstract Database at http://www.ampainsoc.org/abstract/2003/data/index.html. (Poster #915)); Czobor P., Stark J., Beer G., Brown J., Sunshine A., Konery S., Turpin M., Olson N., Otero A., Lippa A., Beer B.: A two center double-blind, placebo-controlled randomized study of DOV 220,075 (bicifadine) SR and Tramadol 100 mg in the treatment of post-operative dental pain. The Journal of Pain, 2004: 5(1), Supplement 1, p 59. Presented at the Joint APS and Canadian Pain Society Annual Meeting (23rd APS Annual Scientific Meeting) May 6-9, 2004, Vancouver, BC Canada. American Pain Society Abstract Database at http://www.ampainsoc.org/abstract/2004/data/index.html (Poster #801); each incorporated herein by reference). However, these findings did not correlate directly to an actual period of analgesic efficacy of bicifadine for treating acute dental pain. On the contrary, the data relied upon by Czobor et al. to suggest a 6 hour or 12 hour efficacy period for bicifadine in acute pain studies were principally comprised of pain ratings assessed much earlier, at 1-4 hours post-dose. In contrast, actual periods of therapeutic efficacy of bicifadine for treating acute pain require direct assessment of pharmacokinetic and pain data throughout a full test period to reliably determine efficacy, and such determinations are further refined by analysis of rescue medication use by study subjects. When these methods were applied to assess the duration of efficacy of bicifadine for treating acute pain, it was determined that patients administered bicifadine SR tablets in standard test dosage amounts (e.g., 200 mg, or 400 mg bicifadine SR tablets) did not show sustained, therapeutically-effective plasma levels of the drug for alleviating acute pain for periods substantially longer than about six hours, or at most about eight hours (see, e.g., Stern et al., “Relationship Between Plasma Bicifadine Levels and Analgesic Effect in a Dental Pain Model, Abstract #314-P291 presented at the 11th World Congress on Pain, Sydney, Australia, Aug. 21-26, 2005, incorporated herein by reference). These findings correlated with a positive dose-dependent relationship for both the pharmacokinetics (AUC, Cmax) of bicifadine and the pharmacodynamic measures of efficacy of bicifadine for treating acute dental pain. Plasma bicifadine levels >1000 ng/ml were associated with the greatest pain relief, and drug levels between 500-1000 ng/ml were associated with significant analgesic efficacy. However, lower plasma drug levels of 500 ng/ml or less were not associated with significant analgesic effects. These data strongly indicated that effective treatment of acute pain using bicifadine would require at least three times daily (tid) dosing, or four times daily (qid) dosing for sustained efficacy.

These conclusions are further substantiated by the use of rescue medication by subjects in the Stem et al. acute dental pain studies. Table I below summarizes information on the use of rescue medication for patients who took rescue medication among the various treatment groups in the clinical trial reported by Stern et al. (id.) A nonparametric analysis (Median Test) was conducted to evaluate the median latency to rescue medication. The Median Test showed that the difference among the five treatment groups (single dose of 200 mg, 400 mg or 600 mg bicifadine SR tablet, Tramadol 100 mg, or Placebo) did not reach statistical significance (Chi-square=4.7, df=4, P=0.32).

TABLE 1
Median Time to Use of Rescue Medication

| | |
|---|---|
| Used Rescue | Median Time to |
| Medication (N) | Rescue Medication |

| Treatment Group* | Yes | No | (hrs) |
|---|---|---|---|
| Bicifadine SR 200 mg | 98 | 9 | 2.08 |
| Bicifadine SR 400 mg | 97 | 12 | 2.32 |
| Bicifadine SR 600 mg | 96 | 11 | 2.48 |
| Tramadol 100 mg | 85 | 24 | 2.45 |
| Placebo | 103 | 5 | 2.08 |

Source: Table 10c (Appendix 16.1.9)
No significant difference among treatments

Survival analysis (Kaplan-Meier method, 95% confidence) was performed to compare the treatment groups with regard to time-to-rescue medication. For the purpose of this analysis, patients who did not take rescue medication until the end of the follow-up period were treated as censored observations. The analysis yielded a statistically significant difference among the treatment groups (Log-Rank test, Chi-square=26.9, df=4, P=0.0001). Subsequent pair-wise comparisons indicated that subjects receiving the bicifadine SR 400-mg (Log-Rank test, Chi-square=9.3, df=1, P=0.002), bicifadine SR 600-mg (Log-Rank test, Chi-square=12.4, df=1, P=0.0004), and tramadol 100-mg treatments (Log-Rank test, Chi-square=18.7, df=1, P=0.0001) were significantly less likely to use rescue medication than study subjects receiving placebo. In view of the foregoing evidence, the present disclosure documenting efficacy of a reduced, bi-daily or less frequent dosing regimen of bicifadine to yield effective treatment of chronic pain are unexpected. The extended duration of treatment efficacy of preferred dosage amounts of bicifadine identified herein does not accord with the findings from the previously-published acute pain studies, nor with the pharmacokinetic data generated from these and related studies. It is a surprising benefit, therefore, that a dosing regimen consisting of only one or two doses of bicifadine effectively alleviates symptoms of chronic pain over an extended period. The extended period of efficacy of the novel compositions and dosage forms of the invention provide significant relief of chronic pain symptoms over a period of at least 8 hours, or at least 12 hours, often at least 18 hours, and up to 24 hours or longer.

The novel dosing methods of the invention for treating chronic pain are not limited to sustained release formulations of bicifadine. Rather, within this aspect of the invention it is contemplated that all delivery modalities can be enlisted to achieve the unexpected therapeutic benefits identified herein attending a reduced dosing regimen of bicifadine, coordinately administered with a NSAID, for treating chronic pain. Thus, in certain embodiments, immediate release formulations of bicifadine may be employed within the subject dosing methods to achieve an unexpected duration of activity for alleviating symptoms of chronic pain.

Within these distinct aspects of the invention, methods for treating chronic pain comprising once daily or twice daily dosing of bicifadine to subjects will employ a once daily or twice daily effective amount of bicifadine coordinately administered with a NSAID (which may be administered on the same dosing schedule as the bicifadine, or on an independent dosing schedule, to yield a combinatorially effective treatment regimen). Effective dosage amounts of bicifadine in this context will typically be between about 25 to 1800 mg, often between about 50 to 1200 mg, more often between about 75 to 1000 mg, or 100 to 600 mg, and in exemplary embodiments between about 200 to 400 mg, or 100 to 200 mg.

Although the novel methods of the invention providing effective treatments for chronic pain using bi-daily or less frequent dosing of bicifadine are not dependent on use of the sustained release (SR) compositions and dosage forms described herein, it will often be advantageous to formulate the bicifadine in an SR dosage form using a sustained release vehicle, matrix, binder or coating material according to the teachings herein. Thus, in certain aspects of the invention, methods for treating chronic pain involving bi-daily or less frequent dosing of bicifadine will employ a SR dosage form of bicifadine that yields extended the release kinetics and lower side effect profile compared to an immediate release (IR) dosage form of bicifadine.

Within exemplary embodiments, SR dosage forms of bicifadine useful for treating chronic pain on a once or twice daily dosing schedule will provide a mean maximum plasma concentration (Cmax) of the active therapeutic agent in a treatment subject which is less than about 80% of a Cmax provided in a control subject after administering the same amount of the active agent in an IR formulation. In related embodiments the SR dosage forms of bicifadine for treating chronic pain yield an Area Under the Curve (AUC) of the active therapeutic agent in a treatment subject which is less than about 80% of an AUC provided in a control subject administered the same amount of bicifadine in an IR formulation. In additional related embodiments for treating chronic pain, a SR dosage form as contemplated herein will yields a Cmax and an AUC of the active therapeutic agent in a treatment subject which are each, respectively, less than about 80% of a Cmax and an AUC provided in a control subject following administration of the same amount of the active agent in an IR formulation.

In yet additional embodiments, SR dosage forms of bicifadine useful for treating chronic pain on a once or twice daily dosing schedule will exhibit an in vitro dissolution profile wherein about 5% to about 35% of the bicifadine is dissolved within 30 minutes, measured in a <711> dissolution test, Apparatus 1, USP 28, 2005, at 37.0° C.±0.5° C., using 900 ml 0.05M potassium phosphate monobasic buffer pH 6.8 and a basket or paddle speed of 75 rpm. In related embodiments, the SR dosage form will exhibit an in vitro dissolution profile wherein about 15% to about 40% of the bicifadine is dissolved within 1 hour according to the foregoing test parameters. In other related embodiments the SR dosage form of bicifadine will exhibit an in vitro dissolution profile wherein about 25% to about 60% of the bicifadine is dissolved within 2 hours according to these test parameters. In additional embodiments the SR dosage form of bicifadine will exhibit an in vitro dissolution profile wherein about 50% to about 80% of the bicifadine is dissolved within 4 hours. In yet additional embodiments the SR dosage form of bicifadine will exhibit an in vitro dissolution profile wherein about 70% to about 90-100% of the bicifadine is dissolved within 8 hours. In still other embodiments, the SR dosage form will exhibit an in vitro dissolution profile wherein about 75% to about 100% of the bicifadine is dissolved within 12 hours. In alternate embodiments, the SR dosage form will exhibit an in vitro dissolution profile wherein about 80% to about 100% of the bicifadine is dissolved within 24 hours. In this context, SR bicifadine formulations which have a more extended dissolution profile will yield more extended in vivo release kinetics, such that extended in vivo release will provide effective therapeutic levels achieved by only once or twice daily dosing that is sustained for a period of at least about 18 hours, and up to 24 hours, or longer.

According to the foregoing description, a sustained release (SR) formulation of bicifadine can have any degree, or any profile, of sustained, delayed, or staged release above what is considered an “immediate release” (IR) profile or formulation. As used herein, an IR formulation is characterized has having no significant additives or barriers to retard release of the drug. For comparative purposes, an IR bicifadine formulation is any formulation, such as a powder, encapsulated powder or granular preparation, or compressed tablet, which releases in a standard in vitro dissolution study, or in a conventional in vivo pharmacokinetic study, at least 75% of the drug, usually more than about 80%, and often greater than 95% of the drug, within 5-10 minutes (e.g., following onset of dissolution testing or after ingestion). Often, 95% or more of the drug in an IR formulation will dissolve in vitro, or in the gastrointestinal tract of a subject, within the first 5-10 minutes. While it will be readily apparent to the skilled artisan how to distinguish between SR and IR formulations of bicifadine, additional discussion and exemplary embodiments of SR and IR bicifadine formulations are provided, for example, in U.S. patent application Ser. No. 11/260,887, filed Oct. 26, 2005, U.S. patent application Ser. No. 10/621,435, filed Jul. 17, 2003; and U.S. Provisional Application No. 60/399,852, filed Jul. 31, 2002, each of which disclosures is incorporated herein by reference for all purposes.

SR formulations of bicifadine for use within the invention yield surprisingly lower side effects in patients compared to IR bicifadine formulations, as shown in Table 2 below.

TABLE 2
Side Effect Profiles of Bicifadine SR and IR Formulations

| Event | Dosage Form | Placebo | | 400-599 mg | | 600-799 mg | | ≧800 mg | |
|---|---|---|---|---|---|---|---|---|---|
| Euphoria | IR | | (1.7%) | | (80.0%) | | (63.6%) | | (90.0%) |
| | SR | | (0.0%) | | (1.3%) | | (4.6%) | | (2.0%) |
| | p-Value | 0.217 | | <0.001 | | | | <0.001 | |
| Dizziness | IR | | (0.0%) | | (60.0%) | | (60.0%) | | (70.0%) |
| | SR | | (7.3%) | | (1.3%) | | (11.7%) | | (7.0%) |
| | p-Value | <0.001 | | <0.001 | | 0.0119 | | <0.001 | |
| Sleepiness/Drowsiness | IR | | (0.0%) | | (80.0%) | | (9.1%) | | (10.0%) |
| | SR | | (5.3%) | | (4.2%) | | (10.7%) | | (3.3%) |
| | p-Value | <0.001 | | <0.001 | | 1.0000 | | 0.3075 | |
| Nausea | IR | | (0.0%) | | (20.0%) | | (18.2%) | | (80.0%) |
| | SR | | (14.9%) | | (24.7%) | | (32.5%) | | (10.2%) |
| | p-Value | <0.001 | | 1.0000 | | 0.5119 | | <0.001 | |
| Mydriasis | IR | | (0.0%) | | (90.0%) | | (30.0%) | | (100%) |
| | SR | | (0.3%) | | (4.9%) | | (11.8%) | | (0.4%) |
| | p-Value | 1.0000 | | <0.001 | | 0.1142 | | <0.001 | |
| Headache | IR | | (0.0%) | | (20.0%) | | (27.3%) | | |
| | SR | | (8.5%) | | (10.9%) | | (13.2%) | | |
| | p-Value | <0.001 | | 0.3082 | | 0.1810 | | | |

The data presented in Table 2 clearly demonstrate that for similar daily doses of bicifadine SR and IR formulations, there is a marked and unexpected decrease in the occurrence of specific adverse events elicited by the SR formulation in comparison to the IR formulation.

In more detailed embodiments of the invention for treating chronic pain using a once daily or twice daily bicifadine dosing regimen, where a SR dosage form is selected, the sustained release vehicle, matrix, binder, or coating material, will often comprise a sustained release polymer. Exemplary sustained release polymers in this context include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as SR vehicles, matrices, binders, or coatings within the compositions and dosage forms of the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-co-glycolic acid) (D PLGA) and poly(L-lactic acid-co-glycolic acid) (L PLGA). Other biodegradable or bioerodable polymers for use within the invention include such polymers as poly(ε-caprolactone), poly(ε-aprolactone-CO-lactic acid), poly(ε-aprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly(ester ureas), poly(2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms comprise bicifadine coated on a polymer substrate. The polymer can be an erodible or a nonerodible polymer. The coated substrate may be folded onto itself to provide a bilayer polymer drug dosage form. For example bicifadine can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the biocrodible dosage form erodes at a controlled rate to dispense the bicifadine over a sustained release period. Representative biodegradable polymers for use in this and other aspects of the invention can be selected from, for example, biodegradable poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, bicifadine is loaded into a polymer that releases the drug by diffusion through a polymer, or by flux through pores or by rupture of a polymer matrix. The drug delivery polymeric dosage form comprises the bicifadine contained in or on the polymer. Representative polymers for manufacturing such SR dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, polyalginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, compositions and dosage forms comprise bicifadine incorporated with or contained in beads that on dissolution or diffusion release the bicifadine over an extended period of hours, for example over a period of at least 6 hours, over a period of at least 8 hours, over a period of at least 12 hours, or over a period of up to 24 hours or longer. The drug-releasing beads may have a central composition or core comprising bicifadine and a pharmaceutically acceptable carrier, along with one or more optional excipients such as a lubricants, antioxidants, dispersants, and buffers. The beads may be medical preparations with a diameter of about 1 to 2 mm. In exemplary embodiments the beads are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14.sup.th ed, pp 1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In other embodiments of the invention, the dosage form for delivering bicifadine may comprise a plurality of tiny pills or mini-tablets. The tiny pills or mini-tablets provide a number of individual doses for providing various time doses for achieving a SR drug delivery profile over an extended period of time, e.g., up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of bicifadine (e.g., a dose of about 10 ng, 0.5 mg, 1 mg, 1.2 mg, 1.4 mg, 1.6 mg, 5.0 mg, etc.) The tiny pills and mini-tablets may further comprise a release rate-controlling wall formed by such materials as a triglyceryl ester (e.g., selected from the group consisting of glyceryl tristearate, glyceryl monostearate, glyceryl dipalmitate, glyceryl laureate, glyceryl didecenoate and glyceryl tridenoate). Other wall forming materials can include polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising bicifadine. In one exemplary embodiment, solid microparticles of bicifadine are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in SR compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing SR compositions and dosage forms comprising bicifadine. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for SR delivery of bicifadine (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, an osmotic dosage form of bicifadine is provided which comprises a semipermeable wall that surrounds the drug. In use within a patient, the osmotic dosage form comprising a homogenous composition imbibes fluid through the semipermeable wall into the dosage form in response to the concentration gradient across the semipermeable wall. The bicifadine in the dosage form develops osmotic energy that causes the drug to be administered through an exit from the dosage form over a prolonged period of time up to 24 hours (or even in some cases up to 30 hours) to provide controlled and sustained drug release. These delivery platforms can provide an essentially zero order delivery profile as opposed to the spiked profiles of immediate release (IR) formulations. Other osmotic dosage forms useful within the invention are described, for example, in U.S. patent application Ser. No. 11/438,909, filed May 22, 2006; U.S. patent application Ser. No. 11/260,887, filed Oct. 26, 2005; and U.S. Pat. Nos. 3,845,770; 3,916,899; 4,063,064; 4,088,864; 4,816,263; 4,200,098; and 4,285,987, each of which disclosures are incorporated herein by reference.

Within other aspects of the invention, microparticle, microcapsule, and/or microsphere drug delivery technologies can be employed to provide SR delivery of bicifadine within the compositions, dosage forms and methods of the invention. Various methods are known for encapsulating drugs within a biocompatible, biodegradable wall-forming material (e.g., a polymer)—to provide sustained or delayed release of the drug. In these methods, the drug is typically dissolved, dispersed, or emulsified in a solvent containing the wall forming material. Solvent is then removed from the microparticles to form the finished microparticle product. Examples of conventional microencapsulation processes for use within the invention are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral SR administration of bicifadine. Exemplary coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of the bicifadine by providing one or more barrier layers, which may be located entirely surrounding the drug, between layers of a multi-layer solid dosage form, and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticiser such as hydrogenated castor oil. The barrier layer may also include conventional binders, fillers, lubricants and compression acids etc such as Polyvidon K30 (trade mark), magnesium stearate, and silicon dioxide.

Additional enteric coating materials for mediating sustained release of bicifadine include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention include coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of bicifadine include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of bicifadine is provided by formulating the drug in a dosage form comprising a multi-layer tablet or other multi-layer or multi-component dosage form. In exemplary embodiments, the bicifadine is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of bicifadine are described, for example, in International Publications WO 95/20946; WO 94/06416; and WO 98/05305 (each incorporated herein by reference). Other multi-component dosage forms for providing sustained delivery of bicifadine include tablet formulations having a core containing the drug coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of bicifadine is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising bicifadine with a release retarding vehicle, matrix, binder, coating, or excipient which allows for slow release of the drug. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™, Methocel E4M™, Methocel K15M™ and Methocel K100™. Other known release-retarding polymers which may be incorporated within this and other embodiments of the invention to provide a SR composition or dosage form of bicifadine include, hydrocolloids such as natural or synthetic gums, cellulose derivatives other than those listed above, carbohydrate-based substances such as acacia, gum tragacanth, locust bean gum, guar gum, agar, pectin, carageenin, soluble and insoluble alginates, carboxypolymethylene, casein, and proteinaceous substances such as gelatin.

Within other embodiments of the invention, a SR delivery device or system is placed in the subject to mediate SR delivery of bicifadine (see, e.g., Goodson, in “Medical Applications of Controlled Release,” supra, vol. 2, pp. 115-138, 1984; and Langer, 1990, Science 249:1527-1533, each incorporated herein by reference). In other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference).

Within each of the foregoing SR delivery forms, SR formulations, and SR delivery methods, bicifadine may be separately formulated and administered from, or combinatorially formulated and simultaneously delivered with, the selected one or more NSAID drugs. The NSAID(s) can be formulated combinatorially with bicifadine using any of the foregoing SR delivery forms, SR formulations, or SR delivery methods, to mediate sustained release of the NSAID comparable to, or selectably varied with respect to, the delivery profile/sustained release kinetics provided for bicifadine.

The methods of the invention for treating chronic pain in mammalian subjects collectively comprise coordinately administering to a treatment subject combinatorially therapeutically effective amounts of bicifadine and one or more NSAID(s) which are released into the subject (e.g., into a gastrointestinal tract of the subject) and allowed to transit to a target site for delivery (e.g., a blood plasma or other tissue or compartment in the subject). In certain embodiments of the invention, these methods employ an SR bicifadine formulation which results in a mean maximum plasma concentration (Cmax) of bicifadine in the treatment subject which is less than about 80% of a Cmax obtained in a control subject after administration of the same amount of bicifadine in an IR formulation. In other embodiments, the method results in an Area Under the Curve (AUC) of the bicifadine in the treatment subject which is less than about 80% of an AUC obtained in a control subject after administration of the same amount of bicifadine in an IR formulation. In other embodiments, the method results in a Cmax and an AUC of the bicifadine in the treatment subject which are each, respectively, less than about 80% of a Cmax and an AUC obtained in a control subject after administration of the same amount of bicifadine in an IR formulation.

Using the methods and compositions of the invention, targeted conditions and/or symptoms of chronic pain are substantially alleviated or prevented in treatment subjects, without attendant, unacceptable adverse side effects. Typically, subjects treated using the methods and compositions of the invention will exhibit an occurrence and/or severity of one or more targeted conditions or symptoms of chronic pain that is reduced by at least 10%, 20%, 30%, 50% or greater, up to a 75-90%, and even 95% or greater, compared to the occurrence and/or severity of the same one or more side effect(s) observed in placebo-treated control subjects under otherwise equivalent or comparable conditions.

In exemplary embodiments of the invention, subjects presenting with osteoarthritis (OA) coordinately administered bicifadine and one or more NSAIDs will exhibit a 5%, 10%, 20%, 30%, 50% or greater reduction, up to a 75-90%, or 95% or greater, reduction, in one or more symptoms associated with OA as compared to placebo-treated or other suitable control subjects. Treatment subjects may also exhibit a 10%, 20%, 30%, 50% or greater decrease, up to a 75-90%, or 95% or greater, decrease, in the symptoms of one or more conditions associated with or complicated by OA including, but not limited to, pain, stiffness, swelling, tenderness, inflammation, and/or functional disability. In functionally disabled subjects, the coordinate administration of bicifadine and one or more NSAID(s) will often yield at least a 10%, 20%, 30%, 50% or greater increase, up to a 75-90%, or 95% or greater increase, in one or more functional/activity indices characterizing their disability (e.g., increased mobility or flexibility).

For determining therapeutic efficacy of the compositions, dosage forms and methods of the invention for treating conditions or symptoms of acute and/or chronic pain in human subjects, there is a variety of useful pain assessment models, assays and scoring systems known in the art. Exemplary methods and tools for assessing efficacy of compositions and methods of the invention for treating chronic pain, such as chronic low back pain (CLBP) include the Pain Severity Rating (PSR), test; the Short-Form McGill Pain Questionnaire (SF-MPQ); and the Roland-Morris Disability Questionnaire. An exemplary PSR test uses a 100 mm visual analogue scale (VAS) to provide a patient pain severity rating, wherein patients are instructed to draw a vertical line on the scale to indicate the amount of low back pain they have experienced over the past 48 hours, from “no pain” to “worst pain imaginable”. Study professionals measure the distance in mm (0-100) from the left side of the scale to the patient's vertical mark and record this number as the PSR value. The SF-MPQ rates the intensity of 15 sensory and affective components of pain and includes VAS and categorical scales to rate present overall pain intensity (see, e.g., Melzack R. The short-form McGill Pain Questionnaire. Pain 30:191-197, 1987). The SF-36 Health Survey is a generic quality of life instrument which has 36 items covering eight domains: physical functioning, role-physical, bodily pain, general health, vitality, social functioning, role-emotional, and mental health (see, e.g., Ware J E, Snow K K, Kosinski M, Gandek B. SF-36R Health Survey Manual and Interpretation Guide. Boston, Mass.: New England Medical Center, The Health Institute, 1993). Each of these indices or parameters can be measured to determine efficacy of the methods and compositions of the invention. For additional pain assessment methods and tools useful for determining efficacy of the compositions, dosage forms and methods of the invention, see, e.g., Strand et al., Back Performance Scale for the assessment of mobility-related activities in people with back pain. Phys Ther. 82:1213-1223, 2002; Linton et al., Int. J. Beh. Med. 7(4):291-304, 2000; and Hsieh et al., J. Manipulative Physiol. Ther. 15(1):4-9, 1992 (each incorporated herein by reference). Using such methods, the efficacy of bicifadine for treating acute pain has been demonstrated in human clinical trials to assess efficacy of bicifadine for treating acute, nociceptive pain following dental surgery—including trials testing activity of bicifadine HCl in side-by-side comparisons against, for example, opiates (see, e.g., Czobor P., et al., supra, 2003; Czobor P., et al., supra, 2004; and U.S. Pat. Nos. 4,231,935 and 4,196,120, each incorporated herein by reference). Additional discussion of bicifadine dosing, formulation, and other subjects related to the treatment of CLBP, and in particular to the use of functional indices to assess dosing, formulation, efficacy, etc., is provided, for example, in U.S. patent application Ser. No. 11/438,909, filed May 22, 2006; U.S. patent application Ser. No. 11/260,887, filed Oct. 26, 2005, U.S. patent application Ser. No. 10/621,435, filed Jul. 17, 2003; and U.S. Provisional Application No. 60/399,852, filed Jul. 31, 2002, each of which disclosures is incorporated herein by reference for all purposes.

In certain embodiments of the invention, subjects treated effectively using the methods and compositions described herein will exhibit an improvement, decreased occurrence, remission, or enhancement in a functional or activity-based disability or quality of life measure or score associated with a targeted condition or symptom of chronic pain. Illustrative functional/disability indices in this context can be determined, for example, by evaluating a subject's difficulty when going down stairs, when going up stairs, when getting up from a sitting position, while standing, when bending to the floor, when walking on a flat surface, when getting in or out of a vehicle, while shopping, when putting on socks or other clothing, when getting in or out of the bathtub, etc. In exemplary embodiments, one or more such functional indices of impairment or disability measures in treated patients will be reduced by at least 10%, 20%, 30%, 50% or greater, up to a 75-90%, and even 95% or greater, compared to the occurrence and/or severity of the same one or more functional indices of impairment or disability measures in placebo-treated control subjects under otherwise equivalent or comparable conditions. For example, patients treated for chronic pain using bicifadine and one or more NSAID(s) can be assessed to determine a “baseline” functional disability index or score prior to treatment, and treated subjects will exhibit an improvement (in terms of increased function, decreased disability, improved activity and/or other functional/quality of life measures) of at least 10%, 20%, 30%, 50% or greater, up to a 75-90%, and even 95% or greater improvement compared to their baseline disability index or score. In more detailed embodiments, patients treated according to the methods and compositions of the invention will show at least a 10%, 20%, 30%, 50% or greater, up to a 75-90%, and even 95% or greater, improvement in a disability rating based on functional/activity measures, for example as embodied in the Roland-Morris Disability Questionnaire, and/or SF-36 Health Survey. Any one or more disability indices may be reduced (corresponding to one or more enhanced functional/activity measures) in different patient populations or using different formulations or treatment protocols according to the invention. Typically, multiple disability indices will be reduced, corresponding to enhancement of one or more functional/activity measures, in treated patients. In illustrative embodiments, subjects treated for chronic pain using coordinate administration of bicifadine and one or more NSAID(s) will exhibit an improvement or decreased occurrence of one or more disability indices, corresponding to enhancement or reversal of one or more functional/activity measures, by at least 10%, 20%, 30%, 50% or greater, up to a 75-90%, and even 95% or greater, identified in the Roland-Morris Disability Questionnaire (RDQ) compared to the occurrence and/or severity of the same one or more functional indices of impairment, or disability measures, in placebo-treated control subjects under otherwise equivalent or comparable conditions. In certain embodiments, improvement in a comprehensive disability or functional/activity measure (e.g., an overall RDQ score) will be observed, for example a 10%, 20%, 30%, 50% or greater, up to a 75-90%, and even 95% or greater reduction in an RDQ score based on the following RDQ format for assessing functional/activity impairment in CLBP patients.

Roland-Morris Disability Questionnaire

Because of My Back Pain Today: (Mark each numbered item YES or NO)

1. I stay at home most the time because of my back.

2. I change position frequently to try to get my back comfortable.

3. I walk more slowly than usual because of my back.

4. Because of my back, I am not doing any of the jobs that I usually do around the house.

5. Because of my back, I use a handrail to get upstairs.

6. Because of my back, I lie down to rest more often.

7. Because of my back, I have to hold on to something to get out of an easy chair.

8. Because of my back, I try to get other people to do things for me.

9. I get dressed more slowly that usual because of my back.

10. I only stand up for short periods of time because of my back.

11. Because of my back, I try not to bend or kneel down.

12. I find it difficult to get out of a chair because of my back.

13. My back is painful almost all the time.

14. I find it difficult to turn over in bed because of my back.

15. My appetite is not very good because of my back pain.

16. I have trouble putting on my socks (or stockings) because of the pain in my back.

17. I only walk short distances because of my back pain.

18. I sleep less well because of my back.

19. Because of my back pain, I get dressed with help from someone else.

20. I sit down for most the day because of my back.

21. I avoid heavy jobs around the house because of my back.

22. Because of my back pain, I am more irritable and bad tempered with people than usual.

23. Because of my back, I go upstairs more slowly than usual.

24. I stay in bed most of the time because of my back.

The methods and compositions of the invention that treat subjects with chronic pain by eliciting an improvement in, or reduced occurrence of, one or more disability indices, or by enhancing one or more functional/activity measures, may yield a corresponding decrease in pain symptoms in treated patients, but they may alternatively achieve the indicated therapeutic benefit indirectly without a direct, or at least directly proportionate, effect of alleviating pain in treated subjects. Thus, the subject methods and compositions that result in reduced disability/improved function may or may not correlate directly, or in all subjects, with a commensurate therapeutic benefit expressed in terms of reduced pain symptoms (e.g., as evinced by VAS or SF-MPQ scores).

In one embodiment of the invention employing bicifadine coordinately administered with one or more NSAID(s) to treat OA, relief of disability in treated patients may be measured, for example, using the Western Ontario McMaster Arthritis scale (WOMAC) or one of its subscales (in other words, the pain, stiffness, or physical function subscales of the WOMAC Osteoarthritis Index). Any suitable version of the WOMAC Osteoarthritis Index may be used, including, for example, Version 3.0 or Version 3.1. The WOMAC Osteoarthritis Index is available in Likert and Visual Analog scaled formats, either of which may be employed in the present methods. WOMAC values can be employed for the diagnosis, prognosis, and monitoring of OA in subjects, as well as for determining efficacy of OA treatment methods and compositions of the invention. Alternatively or additionally, therapeutic efficacy in this context can be assessed using such tools as the Australian/Canadian (AUSCAN) Osteoarthritis Hand Index or the Osteoarthritis Global Index (OGI), the Lequesne index, activities of daily living index (ADL), Knee injury and Osteoarthritis Outcome Score (KOOS), or Hip disability and Osteoarthritis Outcome Score (HOOS). Several additional performance-based tests of functional disability are known in the art and can also be utilized for determining dosing and/or assessing efficacy of the treatment methods and compositions of the invention. In the case of OA, additional tools in this regard include, but are not limited to, ACR Clinical Classification Criteria for Osteoarthritis of the Hip, ACR Clinical Classification Criteria for Osteoarthritis of the Hand, Radiographic Grading of Subtalar Osteoarthritis, Grades of Kellgren and Lawrence for Radiological Appearance of Osteoarthritis, Risk Factors of Dougados et al. for Significant Radiological Progression of Hip Osteoarthritis, Criteria of Altman for the Diagnosis of Idiopathic Osteoarthritis of the Knee Using Clinical Data, Criteria of Altman for the Diagnosis of Idiopathic Osteoarthritis of the Knee Using Clinical and Laboratory Data, Criteria of Altman for the Diagnosis of Idiopathic Osteoarthritis of the Knee Using Clinical and X-Ray Data, Recursive Partitioning Tree of Altman for the Diagnosis of Osteoarthritis of the Knee Using Clinical Data, Recursive Partitioning Tree of Altman for the Diagnosis of Osteoarthitis of the Knee Using Clinical and Laboratory Data, Recursive Partitioning Tree of Altman for the Diagnosis of Osteoarthritis of the Knee Using Clinical, Laboratory and X-Ray Data, Radiographic Grading Scheme of Lane et al. for Osteoarthritis Involving the Hip, Hand or Spine, Therapeutic Response in Osteoarthritis Using the Criteria of the OARSI (Osteoarthritis Research Society International), and ACR Clinical Classification Criteria for Osteoarthritis of the Knee.

The pharmaceutical compositions of the present invention may be administered by any means that achieves the intended therapeutic or prophylactic purpose. Suitable routes of administration for delivering bicifadine and one or more NSAID(s) include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

In certain embodiments the invention provides combinatorial anti-chronic pain formulations comprising bicifadine and a NSAID. Within such combinatorial formulations, bicifadine and a NSAID will be present in a combined formulation in effective amounts, alone or in combination. In certain embodiments, bicifadine and the NSAID(s) will each be present in an amount that is individually therapeutic (i.e., in an individual dosage which will alone elicit a detectable anti-chronic pain response in the subject). Alternatively, a combinatorial formulation may comprise one or both of the bicifadine and NSAID(s) in sub-therapeutic individual dosage amount(s), wherein the combinatorial formulation comprising both bicifadine and the NSAID(s) features a combined dosage of both drugs that is collectively effective in eliciting an anti-chronic pain response. Thus, one or both of the bicifadine and a NSAID may be present in the formulation, or administered in a coordinate administration protocol, at a sub-therapeutic dose, but collectively in the formulation or method they will elicit a detectable anti-chronic pain response (e.g., to alleviate pain or disability) in the subject.

To practice the coordinate administration methods of the invention, bicifadine and a NSAID compound are administered in a coordinate treatment protocol. The coordinate administration may be done simultaneously or sequentially in either order, or separately at different times of day and there may be a time period while only one or both (or all) active therapeutic agents, individually and/or collectively, exert their therapeutic effect. Often the coordinate administration of bicifadine and one or more NSAID(s) will yield an enhanced therapeutic response beyond the therapeutic response elicited by either or both bicifadine and the NSAID compound or compounds alone. As a result, the administration of bicifadine with a NSAID as contemplated herein will result in an effective therapeutic response using lesser amounts of either or both drug(s), thereby reducing or even eliminating adverse side effects, such as toxicity, that may attend log-term administration of one or both drugs alone.

Additional combinatorial formulations and coordinate treatment methods within the invention may employ bicifadine and a NSAID in combination with one or more additional or adjunctive therapeutic agents. Such additional or adjunctive therapeutic agents may include for example, any analgesic including opiate analgesics, topical pain relievers including, but not limited to those containing methyl salicylate, menthol, camphor, eucalyptus or capsaicin, tramadol, acetaminophen, glucosamine, allopurinol, colchicines, demecolcine, oxypurinol, chondroitin, corticosteroids (e.g., glucocorticoids), and hyaluronic acid derivatives (e.g., sodium hyaluronate and hylan G-F20). Within such combinatorial formulations, bicifadine and the NSAID(s) will be present in a combined formulation in effective amounts, alone or in combination.

Since bicifadine and NSAIDs may need to be administered to a patient chronically for the purpose of preventing or treating chronic pain, such as CLBP or OA, combination therapy may involve alternating between administering bicifadine, and one or more NSAID(s) (i.e., alternating therapy regimens between the bicifadine and NSAID(s), e.g., at one week, one month, three month, six month, or one year intervals). Alternating therapy regimens in this context will often reduce or even eliminate adverse side effects, such as toxicity, that may attend long-term use of one or both therapy regimens alone.

The amount, timing and mode of delivery of compositions of the invention comprising an effective amount of a bicifadine and a NSAID composition will be routinely adjusted on an individual basis, depending on such factors as weight, age, gender, and condition of the individual, the severity of the osteoarthritis or related symptoms, whether the administration is prophylactic or therapeutic, and on the basis of other factors known to effect drug delivery, absorption, pharmacokinetics, including, but not limited to, half-life, and efficacy. The precise dose to be employed will also depend on the route of administration, and the seriousness of the disease or disorder, and should be decided according to the judgment of the practitioner and each patient's circumstances. Suitable effective unit dosage amounts of bicifadine utilized in the compositions and methods of the present invention for mammalian subjects may range from about 1 to 1200 mg, 50 to 1000 mg, 75 to 900 mg, 100 to 800 mg, or 150 to 600 mg. In certain embodiments, the effective unit dosage of bicifadine will be selected within narrower ranges of, for example, 10 to 25 mg, 30 to 50 mg, 75 to 100 mg, 50 to 400 mg, 100 to 150 mg, 150 to 250 mg or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of 10 to 25 mg, 30 to 50 mg, 75 to 100 mg, 100 to 200 mg, or 250 to 500 mg, are administered one, two, three, or four times per day. In more detailed embodiments, dosages of 50-75 mg, 100-150 mg, 150-200 mg, 250-400 mg, or 400-600 mg are administered once, twice daily or three times daily. In alternate embodiments, dosages are calculated based on body weight, and may be administered, for example, in amounts from about 0.5 mg/kg to about 30 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

Suitable effective unit dosage amounts of NSAIDs within the compositions and methods of the present invention for mammalian subjects will depend on the NSAID used and its characteristics (e.g., whether short-acting or long-acting). NSAIDs can be administered in recommended clinical dosages, or in subclinical dosages. For example, indomethacin is particularly useful in an amount from about 25 to 75 mg. A typical daily oral dosage of indomethacin is three 25 mg doses taken at intervals during the day. However, daily dosages of up to about 150 mg are useful in some patients. Aspirin will typically be present in tablets or capsules in an amount of between about 250 mg and 1000 mg. Typical daily dosages will be in an amount ranging from 500 mg to about 10 g. However, low dose aspirin present at 20-200 mg (and preferably 40-100 mg) per tablet or capsule may also be used. Ibuprofen may be provided in amounts of 50, 100, 200, 300, 400, 600, or 800 mg. Daily doses should not exceed 3200 mg. Doses of 200 mg-800 mg may be particularly useful when given 3 or 4 times daily. Flurbiprofen is useful in amounts from about from 50 to 100 mg. Daily doses of about 100 to 500 mg, and particularly from about 200 to 300 mg, are usually effective. Ketoprofen is useful in an amount of about 25 to 75 mg. Daily doses of from 100 to 500 mg and particularly of about 100 to 300 mg are typical as is about 25 to 50 mg every six to eight hours. Naproxen in an amount of from 250 to 500 mg. For naproxen sodium, tablets of about 275 or about 550 mg are typically used. Initial doses of from 100 to 1250 mg, and particularly 350 to 800 mg are also used, with doses of about 550 mg being generally preferred. Oxaprozin may be used in amounts in the range of roughly 200 mg to 1200 mg, with about 600 mg being preferred. Daily doses of 1200 mg have been found to be particularly useful and daily doses should not exceed 1800 mg or 26 mg/kg. Etodolac is useful in amounts of 200 mg to 400 mg. Useful doses for acute pain are 200-400 mg every six-eight hours, not to exceed 1200 mg/day. Patients weighing less than 60 kg are advised not to exceed doses of 20 mg/kg. Ketorolac is usefully provided in amounts of about 1-50 mg, with about 10 mg being typical. Oral doses of up to 40 mg, and particularly 10-30 mg/day have been useful in the alleviation of pain. Nabumetone may be provided in amounts of between 500 mg and 750 mg. Daily doses of 1500-2000 mg, after an initial dose of 100 mg, are of particular use. Mefenamic acid is particularly useful in amounts of about 50 mg to 500 mg, with 250 mg being typical. For acute pain, an initial dosage of 1-1000 mg, and particularly about 500 mg, is useful, although other doses may be required for certain patients. Lomoxicam is useful in amounts of about 8 mg to about 16 mg daily for the treatment of arthritis, particularly about 12 mg daily. Celecoxib may be administered in amounts from about 100 mg to about 500 mg or, preferably, from about 100 mg to about 200 mg. Piroxicam may be administered in amounts from about 10 to 20 mg. Rofecoxib will typically be administered in an amount of about 12.5, 25 or 50 mg. The recommended initial daily dosage for the management of acute pain is 50 mg. Meloxicam is typically administered in a daily dose of about 7.5 to about 15 mg for the management of osteoarthritis. Valdecoxib is generally administered in amounts from about 10 mg to about 20 mg. These ranges are provided by way of example. Typical dosage regimens for NSAIDs can be obtained from standard references such as the 2005 Physicians' Desk Reference by Medical Economics. These and other effective unit dosage amounts may be administered in a single dose, or in the form of multiple daily, weekly or monthly doses, for example in a dosing regimen comprising from 1 to 5, or 2-3, doses administered per day, per week, or per month. The dosage amounts described herein refer to total amounts administered; that is, if bicifadine and one or more NSAID(s) are administered, the preferred dosages correspond to the total amount administered. Oral compositions preferably contain about 10% to about 95% of total active ingredients by weight.

Pharmaceutical dosage forms of the bicifadine and a NSAID compound or compounds of the present invention may include carriers, excipients, and other ingredients recognized in the art of pharmaceutical compounding as being suitable for the preparation of dosage units as discussed above. Such additives may include, without limitation, binders, fillers, adjuvants, lubricants, emulsifiers, suspending agents, sweeteners, flavorings, preservatives, diluents, buffers, wetting agents, disintegrants, effervescent agents and other conventional additives and pharmaceutical delivery agents known to those skilled in the art. These additional formulation additives and delivery agents will generally be biologically inactive and can be administered to patients without causing deleterious side effects or interactions with the active drug(s).

Bicifadine and NSAID compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate.

The invention also provides combinatorial pharmaceutical preparations in kit form. Exemplary kits in this context include a first therapeutic agent, bicifadine, in a selected unit dosage form and amount, and a second therapeutic agent comprising one or more NSAID(s) in a selected dosage form and amount. The kits further comprise means for containing the bicifadine and NSAID(s), separately or together, in one or more container(s), bottle(s), package(s) or other containment means. Typically, the kits will include directions for administering the bicifadine and NSAID(s) to treat a chronic pain condition, such as CLBP or OA.

The above disclosure generally describes the present invention. A more complete understanding can be obtained by referring to the following examples. These examples are described solely for purposes of illustration and are not intended to limit the scope of the invention. Although specific terms have been employed herein, such terms are intended for descriptive use and not for purposes of limitation.

### EXAMPLES

Utilizing an accepted, in vivo animal model considered predictive of anti-osteoarthritis (OA) activity in humans, the following examples demonstrate that bicifadine in combination with a prototypical NSAID (ibuprofen) is surprisingly effective in reducing and/or alleviating pain and other OA symptoms, including functional disabilities, in patients with OA.

### Example 1

### Comparative Activity of Bicifadine and Morphine for Treating Monoiodoacetamide (MIA)-Induced Osteoarthritis in Rats

Male Wistar rats (175-200 g) were housed in solid bottom isolator cages, 2-4 rats per cage, with corncob bedding on a 12 hour: 12 hour light:dark cycle. Animals were fed standard rat chow with water available ad libitum.

The rats were anesthetized with 5% volume/volume (“v/v”) isoflurane gas and maintained with 2% v/v isoflurane gas. The anesthetized rats were given a single intra-articular injection of 2 mg of MIA through the infrapatellar ligament of the right knee. MIA was dissolved in physiologic saline and administered in a volume of 50 μL. The contralateral control knee was injected with 50 μL of physiologic saline. Administration of isoflurane gas was discontinued, and the rats became fully conscious about 5 minutes later.

Shifts in hind paw weight distribution from the right to the left hind paws supporting the right (arthritic) and the left (contralateral control) hind leg knee joints were utilized as an index of joint pain and as a measure of compound efficacy. An incapacitance tester (Model 2KG, Linton Instrumentation, UK) was employed for determination of hind paw weight distribution.

The acute dosing paradigm used herein relates to osteoarthritis signs such as mobility and joint function and osteoarthritis symptoms such as joint pain. Rats were induced with MIA as described above. Changes in hind paw weight distribution were determined early on Day 14 post MIA injection to establish a baseline pain reading using an incapacitance tester. The incapacitance tester has a chamber on top with an outwardly sloping front wall that supports a rat's front limbs, and two weight sensing pads, one for each hind paw, that facilitates this determination. Once the baseline was established, rats were then given vehicle, bicifadine in doses of 5, 10, 20, 40 or 60 mg/kg, or 60 mg/kg of morphine via oral gavage (PO). Changes in hind paw weight distribution as an indication of pain were then determined 1 and 2 hours post-administration.

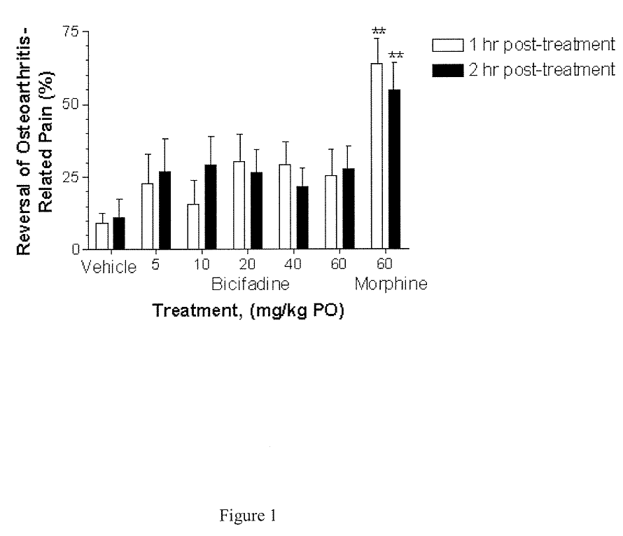

As is shown in FIG. 1, bicifadine had no significant effect on osteoarthritis-related pain induced by MIA. In contrast, morphine, used as a positive control, significantly reduced the pain. (**:P<0.01, Statistically significant differences were determined by one-way ANOVA followed by Dunnett's multiple comparisons procedure).

### Example 2

### Combinatorial Efficacy of Bicifadine and Ibuprofin Demonstrated in Human Osteoarthritis Subjects

23 adult human subjects with clinically confirmed osteoarthritis (OA) of the hip or knee were selected based on (1) a finding of OA through clinical analysis of hip or knee pain for at least six months, (2) stiffness symptoms that persisted for more than 30 minutes (4) crepitus and (5) score of >40 mm on the visual analog scale (VAS). Additionally, radiographic imaging demonstrated the presence of osteophytes in affected joints of the study subjects. (Altman R. Asch E, Bloch D, Bole G, Borenstein D, Brandt K, et al. The American College of Rheumatology criteria for the classification and reporting of osteoarthritis of the knee. Arthritis Rheum 1986; 29:1039-1049) The subjects were randomized to a sequence of 400 mg of bicifadine twice daily for seven days (B), ibuprofen 800 mg twice daily for seven days (I), placebo twice daily for seven days (P), or 400 mg bicifadine and 800 mg ibuprofen twice daily for seven days (B+I) as shown in Table 3. Each of the treatment periods consisted of a seven-day treatment phase. Each of the four treatment periods was separated by a seven-day washout period.

TABLE 3
Treatment sequence

| Group | Treatment | Treatment | Treatment | Treatment |
|---|---|---|---|---|
| Number | Period 1 | Period 2 | Period 3 | Period 4 |
| I | P | B + I | B | I |
| II | B | P | I | B + I |
| III | I | B | B + I | P |
| IV | B + I | I | P | B |

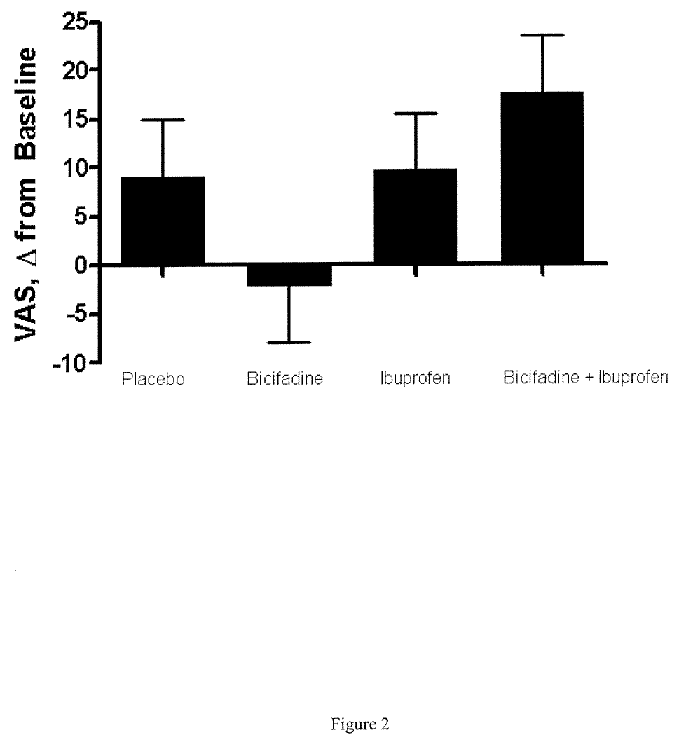

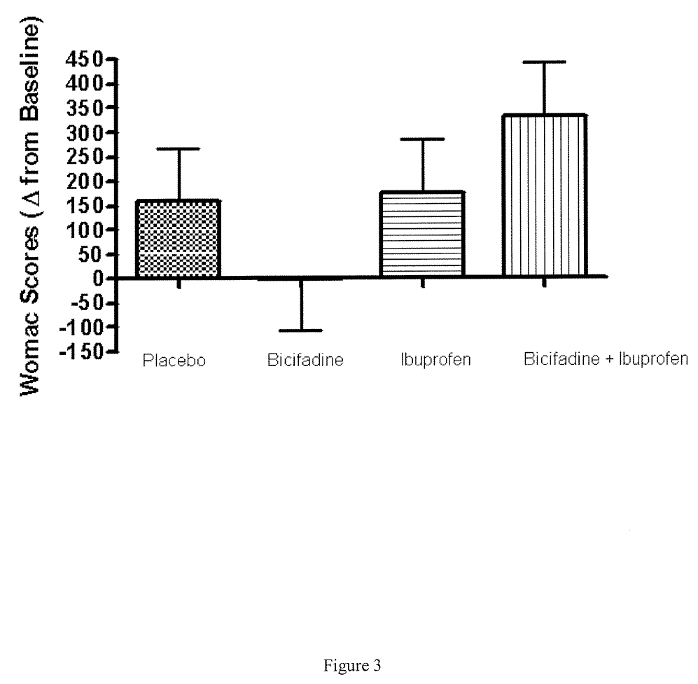

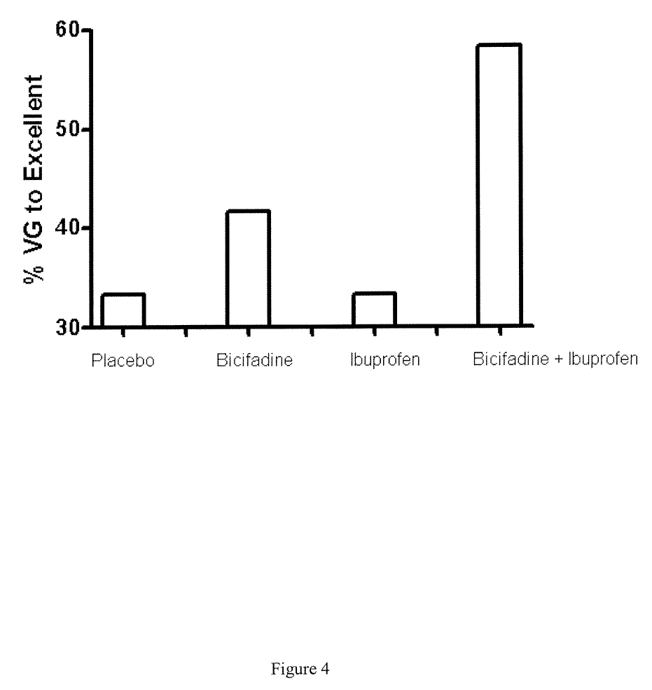

Each patient's pain and function levels was rated just prior to administration of the first dose in each treatment period to establish a baseline and three hours after administration of the final dose of each treatment period. Subjects were evaluated according to the visual analog scale (FIG. 2), the Western Ontario and MacMaster Universities Osteoarthritis Index (WOMAC) (FIG. 3) and a global assessment score (FIG. 4).

For the visual analog scale, each subject was asked the following question: “Draw a vertical line on the scale to indicate the average amount osteoarthritis pain you have in your index hip or knee in the prior 24 h period.
No Pain [______] Worst Pain You Can Imagine 100 mm
   The length of the line from “No Pain” to the vertical line drawn by the patient was then measured to determine the VAS measurement.
   Subjects were also questioned regarding pain severity and pain relief as follows:
   Acute Pain Severity:
   The subjects were asked the following question:
   “How much pain do you have at this time:
      None
      Mild
      Moderate
      Severe”
         Acute Pain Relief:
         The subjects were asked the following question:
   “How much relief do you have from your starting pain:
      None
      A little
      Some
      A lot
      Complete”
         As is shown in FIG. 2, treatment with bicifadine or ibuprofen alone was no different than treatment with placebo in reducing OA pain as measured by the visual analog scale. In contrast, treatment with the combination of bicifadine and ibuprofen resulted in a significant decrease in OA subjects' pain levels.

Subjects were also evaluated according to the Western Ontario and MacMaster Universities Osteoarthritis Index (WOMAC) and asked the following series of questions:

Section A:

How much pain do you have:

1. Walking on a flat surface?

2. Going up or down stairs?

3. At night while in bed?

4. Sitting or lying?

5. Standing upright?

Section B:

6. How severe is your stiffness after first awakening in the morning?

7. How severe is your stiffness after sitting, lying, or resting later in the day?

Section C:

What degree of difficulty did you have with . . .

8. Descending stairs?

9. Ascending stairs?

10. Rising from sitting?

11. Standing?

12. Bending to the floor?

13. Walking on a flat surface?

14. Getting in and out of a car?

15. Going shopping?

16. Putting on socks or stockings?

17. Rising from bed?

18. Taking off socks or stockings?

19. Lying in bed?

20. Getting in and out of the bath?

21. Sitting?

22. Getting on and off the toilet?

23. Heavy domestic duties?

24. Light domestic duties?

As can be seen in FIG. 3, bicifadine and ibuprofen, given individually, were also no better than placebo in improving functioning as determined by the WOMAC osteoarthritis index. However, the combination of bicifadine and ibuprofen improved the functioning of the subjects. (FIG. 3).

Subjects were further evaluated for a global assessment and were asked the following question:

“How would you rate the study medication you received during this treatment period for your osteoarthritis pain?”

No Effect (0); Poor (1); Fair (2); Very Good (3); Excellent (4)

While bicifadine treatment alone was marginally better than treatment with either placebo or ibuprofen alone according to the global assessment score, the combination of bicifadine and ibuprofen was more effective than placebo, ibuoprofen, or bicifadine given individually in alleviating pain and increasing functioning in the patients. (FIG. 4)

As can be seen in FIGS. 2-4, the combination of bicifadine and ibuprofen significantly improved the comfort and alleviated the pain experienced by the subjects than either bicifadine or ibuprofen alone. Thus, bicifadine is unexpectedly effective for treating OA in combination with ibuprofen, and will therefore be useful in combinatorial formulations and coordinate treatment methods with other NSAID(s) for treating OA and other chronic pain conditions.

Although the foregoing invention has been described in detail by way of example for purposes of clarity of understanding, it will be apparent to the artisan that certain changes and modifications may be practiced within the scope of the appended claims which are presented by way of illustration not limitation. In this context it will be understood that this invention is not limited to the particular formulations, process steps, and materials disclosed herein as such formulations, process steps, and materials may vary somewhat. It will also be understood that the terminology employed herein is used for the purpose of describing particular embodiments only, and is not intended to be limiting since the scope of the present invention will be limited only by the appended claims and equivalents thereof. It is further noted that various publications and other reference information have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

We claim:

1. A method for preventing or treating a condition or symptom of chronic pain in a mammalian subject, comprising administering to the subject a first therapeutic agent comprising bicifadine, and a second therapeutic agent comprising a non-steroidal anti-inflammatory drug (NSAID).

2. The method according to claim 1, wherein the bicifadine is racemic bicifadine hydrochloride.

3. The method according to claim 1 wherein the bicifadine is a pharmaceutically acceptable salt of racemic bicifadine.

4. The method according to claim 1, wherein the bicifadine is a (+)-enantiomer of bicifadine.

5. The method according to claim 4, wherein the (+)-enantiomer of bicifadine is administered in a formulation that is substantially free of the (−) enantiomer of bicifadine.

6. The method according to claim 1, wherein the bicifadine is a (−) enantiomer of bicifadine.

7. The method according to claim 6, wherein the (−)-enantiomer of bicifadine is administered in a formulation that is substantially free of the (+)-enantiomer of bicifadine.

8. The method according to claim 1, wherein the bicifadine comprises a bicifadine HCl polymorph form B.

9. The method according to claim 8, wherein the polymorph form B of bicifadine hydrochloride is administered in a formulation that is substantially free of polymorph form A of bicifadine.

10. The method according to claim 1, wherein the NSAID is selected from the group consisting of: salicylates, arylalkanoic acids, N-acylanthranilic acids (fenamic acids), oxicams, coxibs, sulphonanilides, napthylalkanones, acetic acids, propionic acids, sulfonamides, pyrazoles, aminonicotinic acids, pyrazolones, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide.

11. The method according to claim 10, wherein the salicylate is aspirin, aloxiprin, salsalate, choline magnesium trisalicylate, diflunisal, salicylamide, salicylic acid, choline salicylates, magnesium salicylate, sodium salicylate, triethanolamine salicylate, flufenisal, benorylate, or fisalamine.

12. The method according to claim 10, wherein the arylakanoic acid is diclofenac, aclofenac, indomethacin, desoxysulindac or sulindac.

13. The method according to claim 10, wherein the N-arylanthranilic acid is mefenamic acid, flufenamic acid, or meclofenamate sodium.

14. The method according to claim 10, wherein the oxicam is piroxicam, tenoxicam, meloxicam, lomoxicam or tesicam.

15. The method according to claim 10, wherein the coxib is celecoxib, rofecoxib, valdecoxib, parecoxib or etoricoxib.

16. The method according to claim 10, wherein the sulphonanilide is nimesulide.

17. The method according to claim 10, wherein the napthylalkanone is nabumetone.

18. The method according to claim 10, wherein the acetic acid is diclofenac, ibufenac, fenbufen, indomethacin, indoxole, sulindac, etoldac or tolmetin.

19. The method according to claim 10, wherein the propionic acid is oxaprozin, ibuprofen, flurbiprofen, oxaprozin, ketoprofen, naproxen, naproxol, carprofen, fenoprofen, fluprofen or ketorolac.

20. The method according to claim 10, wherein the sulfonamide is trifumidate.

21. The method according to claim 10 wherein the pyrazole is phenylbutazone, aminopyrine, antipyrine, oxyphenbutazone or tetrydamine.

22. The method according to claim 10, wherein the aminonicotinic acid is flunixin.

23. The method according to claim 10, wherein the pyrazolone is phenylbutazone, feprazone or apazone.

24. The method according to claim 1, further comprising administering a tertiary or adjunctive agent to said subject.

25. The method of claim 24, wherein the tertiary or adjunctive agent is selected from NSAIDs, analgesics, opiates, topical pain relievers, corticosteroids, hyaluronic acid derivatives, acetaminophen, tramadol, glucosamine, allopurinol, colchicine, demecolcine, oxypurinol, and chondroitin.

26. The method of claim 25, wherein the topical pain reliever contains methyl salicylate, menthol, camphor, eucalyptus or capsaicin.

27. The method of claim 24, wherein the tertiary or adjunctive agent is an opiate.

28. The method of claim 1, further comprising an adjunctive therapy selected from dietary therapy, exercise, weight loss, heat treatment, cold treatment, acupuncture, joint replacement, osteotomy, arthroscopic lavage and debridement, repositioning of bones, bone fusion, discectomy and spinal fusion.

29. The method of claim 1, wherein said bicifadine is administered in an effective amount between about 0.8 mg to about 15 mg of bicifadine per kg per day.

30. The method of claim 1, wherein the effective amounts of bicifadine comprise between 50 and 800 mg of bicifadine per day.

31. The method of claim 1, wherein the effective amounts of bicifadine comprise between about 50 and 800 mg of bicifadine per day.

32. The method of claim 1, said bicifadine is administered in an effective amount between about 100 and 600 mg of bicifadine per day.

33. The method of claim 1, wherein said bicifadine is administered in an effective amount between about 50 and 400 mg of bicifadine per day.

34. The method of claim 1, wherein the NSAID is ibuprofen.

35. The method of claim 33, wherein the ibuprofen is administered in an effective amount between about 400 mg and 1600 mg of ibuprofen per day.

36. The method of claim 1, wherein the condition or symptom of chronic pain is selected from the group consisting of osteoarthritis pain; rheumatoid arthritis pain; cancer pain; chronic low back pain (CLBP); chronic lumbar pain; chronic cervical pain; chronic fibromyalgia pain; chronic pain from arteriovenuous malformation; arachnoiditis; chronic pain from root avulsion; chronic postthoracotomy pain; and chronic postmastectomy pain of non-neuropathic origin

37. The method of claim 36, wherein the condition or symptom of chronic pain is osteoarthritis pain.

38. The method of claim 36, wherein the condition or symptom of chronic pain is CLBP.

39. A pharmaceutical composition for preventing or treating chronic pain in a mammalian subject comprising bicifadine and a non-steroidal anti-inflammatory drug (NSAID).

40. The composition according to claim 39, wherein the bicifadine comprises racemic bicifadine hydrochloride.

41. The composition according to claim 39, wherein the bicifadine is a pharmaceutically acceptable salt of racemic bicifadine.

42. The composition according to claim 39, wherein the bicifadine is a (+)-enantiomer of bicifadine as a pharmaceutically acceptable salt.

43. The composition according to claim 42, wherein the (+)-enantiomer of bicifadine is administered in a formulation that is substantially free of the (−) enantiomer of bicifadine.

44. The composition according to claim 39, wherein the bicifadine is a (−) enantiomer of bicifadine.

45. The composition according to claim 44, wherein the (−)-enantiomer of bicifadine is administered in a formulation that is substantially free of the (+)-enantiomer of bicifadine.

46. The composition according to claim 39, wherein the bicifadine comprises a bicifadine polymorph form B.

47. The composition according to claim 46, wherein the polymorph form B of bicifadine is administered in a formulation that is substantially free of polymorph form A of bicifadine.

48. The composition according to claim 39, wherein the non-steroidal anti-inflammatory is selected from the group consisting of salicylates, arylalkanoic acids, N-arylanthranilic acids (fenamic acids), oxicams, coxibs, sulphonanilides, napthylalkanones, acetic acids, propionic acids, sulfonamides, pyrazoles, aminonicotinic acids, pyrazolones, benzindopyrine hydrochloride, benzydamine hydrochloride, cinchophen, cintazone, clonixeril, clonixin, diflumidone sodium, dimefadane, fenamole, flutiazin, intrazole, letimide hydrochloride, metazamide, mimbane hydrochloride, molinazole, neocinchophen, nexeridine hydrochloride, nimazole, octazamide, paranylene hydrochloride, proxazole citrate, and tesimide.

49. The composition according to claim 48, wherein the salicylate is aspirin, aloxiprin, salsalate, choline magnesium trisalicylate, diflunisal, salicylamide, salicylic acid, choline salicylates, magnesium salicylate, sodium salicylate, triethanolamine salicylate, flufenisal, benorylate, or fisalamine.

50. The composition according to claim 48, wherein the arylakanoic acid is diclofenac, aclofenac, indomethacin, desoxysulindac or sulindac.

51. The composition according to claim 48, wherein the N-arylanthranilic acid is mefenamic acid, flufenamic acid, or meclofenamate sodium.

52. The composition according to claim 48, wherein the oxicam is piroxicam, tenoxicam, meloxicam, lomoxicam or tesicam.

53. The composition according to claim 48, wherein the coxib is celecoxib, rofecoxib, valdecoxib, parecoxib or etoricoxib.

54. The composition according to claim 48, wherein the sulphonanilide is nimesulide.

55. The composition according to claim 48, wherein the napthylalkanone is nabumetone.

56. The composition according to claim 48, wherein the acetic acids is diclofenac, ibufenac, fenbufen, indomethacin, indoxole, sulindac, etoldac or tolmetin.

57. The composition according to claim 48, wherein the propionic acid is oxaprozin, ibuprofen, flurbiprofen, oxaprozin, ketoprofen, naproxen, naproxol, carprofen, fenoprofen, fluprofen or ketorolac.

58. The composition according to claim 48, wherein the sulfonamide is trifumidate.

59. The composition according to claim 48, wherein the pyrazole is phenylbutazone, aminopyrine, antipyrine, oxyphenbutazone or tetrydamine.

60. The composition according to claim 48, wherein the aminonicotinic acid is flunixin.

61. The composition according to claim 48, wherein the pyrazolone is phenylbutazone, feprazone or apazone.

62. The composition according to claim 39, further comprising a tertiary or adjunctive agent.

63. The composition of claim 39, comprising between about 50 and 800 mg of bicifadine.

64. The composition of claim 39, comprising between about 100 and 400 mg of bicifadine.

65. The composition of claim 39, comprising between about 100 and 200 mg of bicifadine.

66. The composition of claim 39, wherein the non-steroidal anti-inflammatory drug is ibuprofen.

67. The composition of claim 66, comprising between about 400 mg and 1600 mg of ibuprofen.

68. A method for treating a disability or reducing a functional impairment associated with a chronic pain condition in a mammalian subject comprising coordinately administering bicifadine and a non-steroidal anti-inflammatory drug (NSAID) to said subject.

69. The method of claim 68, wherein one or more functional indices of impairment or disability is reduced in treated subjects compared to placebo-treated subjects by at least 20%.

70. The method of claim 68, wherein one or more functional indices of impairment or disability is reduced in treated subjects compared to placebo-treated subjects by at least 50%.

71. The method of claim 68, wherein a baseline functional disability index or score of subjects prior to treatment is improved after treatment by at least 20%.

72. The method of claim 68, wherein a baseline functional disability index or score of subjects prior to treatment is improved after treatment by at least 50%.

73. The method of claim 68, wherein said chronic pain condition is selected from osteoarthritis pain; rheumatoid arthritis pain; cancer pain; chronic low back pain; chronic lumbar pain; chronic cervical pain; chronic fibromyalgia pain; chronic pain from arteriovenuous malformation; arachnoiditis; chronic pain from root avulsion; chronic postthoracotomy pain; and chronic postmastectomy pain of non-neuropathic origin.

74. The method of claim 1, wherein said bicifadine is formulated for oral delivery with a sustained release vehicle, matrix, binder, or coating material

75. The method of claim 74, wherein the sustained release vehicle, matrix, binder, or coating material comprises a sustained release polymer.

76. The method of claim 75, wherein the sustained release polymer is selected from the group consisting of consisting of ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

77. The method of claim 68, wherein said bicifadine is formulated for oral delivery with a sustained release vehicle, matrix, binder, or coating material

78. The method of claim 77, wherein the sustained release vehicle, matrix, binder, or coating material comprises a sustained release polymer.

79. The method of claim 78, wherein the sustained release polymer is selected from the group consisting of consisting of ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.
